(19) United States
(12) Patent Application Publication
(10) Pub. No.: US 2008/0243146 A1
(43) Pub. Date: Oct. 2, 2008

(54) LOCATING AND OCCLUDING VESSELS

(76) Inventors: Todd Sloan, Medway, MA (US); Alfred Intoccia, Nashua, NH (US); Jon T. McIntyre, Newton, MA (US); Kenneth J. Daignault, Holden, MA (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 11/959,895

(22) Filed: Dec. 19, 2007

Related U.S. Application Data
(60) Provisional application No. 60/878,838, filed on Jan. 4, 2007.

(51) Int. Cl. A61B 17/062; A61B 17/04

(52) U.S. Cl. 606/144; 606/228

(57) ABSTRACT

A medical device for blood vessel detection and occlusion includes an elongated member, a sensor, and an occluder. The elongated member includes a distal portion configured and dimensioned for accessing anatomical regions. The distal portion includes a recessed area. The sensor of the device can sense a blood vessel disposed in or near the recessed area. The occluder is operatively associated with the distal portion, and it is configured for at least partially stopping the flow of blood through the blood vessel disposed within the recessed area.

This application claims priority to U.S. Provisional Patent Application No. 60/878,838, filed Jan. 4, 2007, entitled “Locating and Occluding Vessels,” the entire content of which is hereby incorporated by reference.

### TECHNICAL FIELD

The present invention generally relates to medical devices and methods for locating and occluding an anatomical vessel within the body of a human or other mammal.

### BACKGROUND INFORMATION

Uterine fibroids, or myomas (short for leiomyomas), affect more than 30% of women. The terms fibroid and myoma are used interchangeably. Most fibroids do not cause symptoms, and do not require treatment. Fibroids may require treatment, however, if they are growing rapidly, have grown large enough to cause pressure on other organs (such as the bladder), cause abnormal bleeding, or cause problems with fertility, for example.

Therapies have been devised to treat uterine fibroids without hysterectomy. For example, surgical methods (both open, interventional surgery and endoscopic/hysteroscopic surgery) have been developed to destroy fibroids in situ. Myomectomy uses standard or miniature surgical instruments to cut a fibroid away from the uterus. After the fibroid is cut away, the uterine muscle is then sutured back together. Myolysis is a process by which probes are used to focus energy directly into the fibroid to heat the fibroid tissue sufficiently to destroy the fibroid. Laser, radiofrequency, and microwave energies have been used for this purpose.

One treatment for fibroids is called uterine artery embolization. Embolization is a minimally invasive means of blocking the arteries that supply blood to the fibroids. The procedure was first used in fibroid patients in France as a means of decreasing the blood loss that occurs during myomectomy. It was discovered that after the embolization, while awaiting surgery, many patients' symptoms went away and surgery was no longer needed. The blockage of the blood supply caused degeneration of the fibroids and this resulted in resolution of their symptoms without negative impact on the normal myometrium. This has led to the use of the transvaginal clamp, which is left in place on the artery while the patient is under anesthesia, or heavily sedated. The patient has to be kept very still during this period, which can be six hours or more, to ensure that the clamps remain in position.

### SUMMARY OF THE INVENTION

The present invention relates to blood vessel detection and occlusion device, and in particular devices and methods for detecting and occluding a vessel such as a uterine artery of a female human patient.

In one aspect, a medical device according to the invention includes an elongated member, a sensor, and an occluder. The elongated member includes a distal portion configured and dimensioned for accessing anatomical regions. The distal portion includes a recessed area, which can be defined by an arcuate or generally arcuate section of the distal portion. The sensor is disposed on the distal portion within the recessed area, and the sensor is used to sense a blood vessel disposed within or adjacent the recessed area. The occluder is operatively associated with the distal portion, and it is configured for at least partially stopping the flow of blood through the blood vessel disposed within the recessed area.

Embodiments according to this aspect of the invention can include the following features. The occluder can comprise a suture placement mechanism. The suture placement mechanism can comprise a needle deployment and catch mechanism, and the needle deployment and catch mechanism can include a needle carrier, a needle catch, and an actuator. The needle carrier can move a needle out of the distal portion on one side of the recessed area and into the needle catch on the other side of the recessed area, and a suture can be attached to the needle. The needle catch can receive and retain the needle. The actuator can be disposed at the proximal end of the elongated member and can be coupled to the needle carrier. The actuator can comprise a button actuatable by a hand of a user, with a shaft extending from the button. The sensor can be an ultrasound sensor. The sensor may be of another type, such as an audio transmitter and/or receiver (e.g., a microphone) or a pressure transducer, as long as it can be used to locate a blood vessel within the body.

In another aspect, the invention relates to a method of locating and occluding a blood vessel. The method comprises the steps of providing the medical device described above, maneuvering the device so that the blood vessel to be occluded is within the recessed area, and then at least partially stopping the flow of blood through the blood vessel using the medical device.

In this other aspect of the invention, the occluder can comprise a suture placement mechanism, and a suture can be placed around the blood vessel by using the mechanism. In a further aspect of the invention, the sensor comprises an ultrasound sensor that can detect the flow of blood through the blood vessel.

In yet another aspect, the invention relates to a medical device comprising means for accessing anatomical regions of a patient and for receiving at least a blood vessel of the patient, means for sensing the blood vessel, and means for at least partially stopping the flow of blood through the sensed blood vessel.

The occluding means can comprise a suture placement mechanism, and this mechanism can comprise a needle deployment and catch mechanism. The needle deployment and catch mechanism can comprise a needle carrier and a needle catch. A needle can be removably couplable to the needle carrier, and a suture can be attached to the needle. The sensing means can comprise an ultrasound transducer, a microphone, or a pressure transducer, for example.

These and other aspects, embodiments, and features of the invention will become more readily apparent from the following description.

### BRIEF DESCRIPTION OF THE DRAWINGS

Illustrative embodiments according to the invention will be described in detail hereinbelow with reference to the drawings, wherein:

FIG. 1A is a schematic plan view of one embodiment of a vessel detection and occluding device in accordance with the invention;

FIG. 1B is a schematic cross sectional view of a proximal portion of the device of FIG. 1A;

FIG. 1C is a schematic cross sectional view of a distal portion of the device of FIG. 1A;

FIG. 2 is a schematic perspective view of a distal portion of the distal portion of the device of FIG. 1A;

FIG. 3 is a schematic view illustrating the reproductive anatomy of a typical human female patient, including, in particular, the vagina, the uterus, and the left and right uterine arteries; and

FIGS. 4-7 illustrate an exemplary method of locating and occluding a uterine artery in accordance with the present invention using the device of FIG. 1A.

### DESCRIPTION

The following description is provided to illustrate various embodiments of the invention, but the description is not intended to limit the scope of the invention.

The present invention is generally directed to medical devices that can be used to locate and occlude anatomical vessels. An exemplary use for such a device is in treating women for symptomatic fibroid disease. Thus, to illustrate features and aspects of the present invention, among other things, embodiments of the present invention discussed herein are embodiments configured and dimensioned for treating uterine fibroids in female patients. Although features and aspects of the invention make it well suited for this purpose, the invention is not limited only to medical devices for treating fibroids in female patients.

One embodiment of the invention involves a single device for treating uterine disorders, particularly uterine fibroids. The device can locate and at least partially stop the flow of blood through a uterine artery or both uterine arteries using a trans-vaginal, a trans-uterine, a trans-rectal, or a retroperitoneal approach. One advantage is that devices and methods according to the invention may be employed by a patient's gynecologist in the course of regular treatment, avoiding the need for referrals to specialist practitioners and for more radical treatments such as hysterectomy.

In accordance with the invention, ultrasound or other sensing technology (such as a pressure transducer, or an audio receiving and/or transmitting device like a microphone) is incorporated into a minimally-invasive medical device to determine the location or position of a blood vessel. Once the location is determined, the flow of blood through the sensed blood vessel can be at least partially stopped so as to cut off most or all of the blood supply feeding unwanted tissue such as cysts and the like. While it is desirable to stop completely the flow of blood through the vessel completely, partially stopping the flow of blood through the vessel also can result in the starvation and death of unwanted tissue previously fed by blood passing through the vessel. In any event, the occlusion of the blood vessel by either complete or partially stoppage of blood flow through the vessel can be achieved by placing a suture around the vessel and then tightening the suture around the vessel.

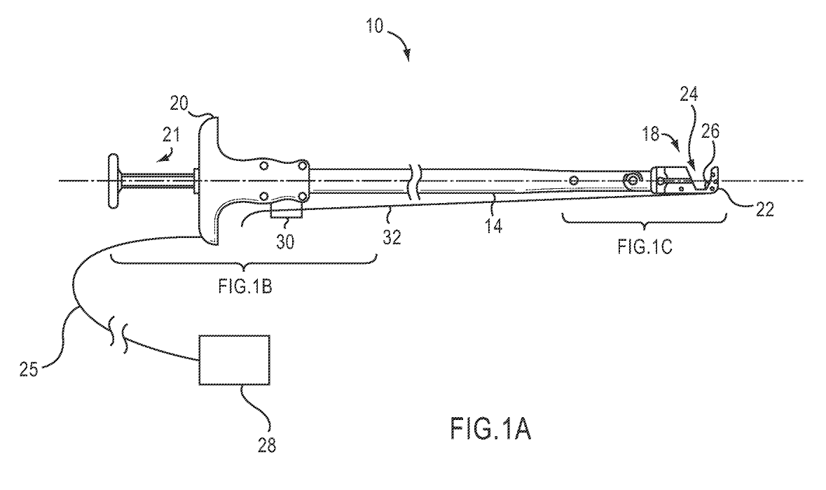

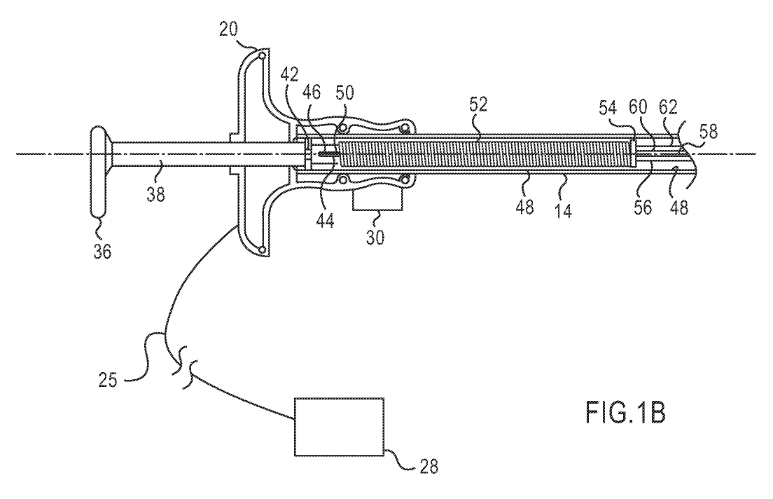

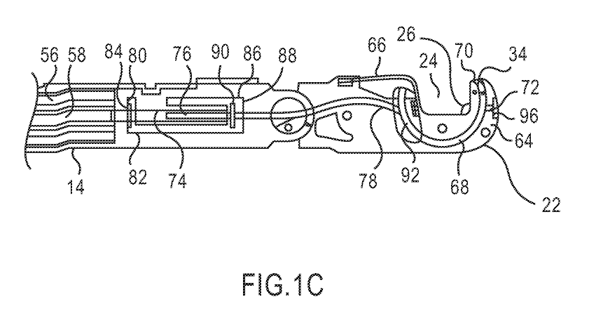

Referring to FIGS. 1A-1C, in one embodiment according to the invention, a device 10 includes a handle 20 extending from a proximal end of an elongated body member 14. The elongated body member 14 includes a distal head portion 18. The elongated body member 14 is mechanically coupled to the handle 20 in this embodiment. The distal head portion 18 includes an arcuate or generally arcuate section 22 defining a recessed area 24 having a generally semi-circular cross sectional profile.

An ultrasound sensor 26 is capable of indicating the proximity of a blood vessel relative to the recessed area 24 by sensing blood flow through the blood vessel when the blood vessel is disposed in or near the recessed area 24. In a typical application where the distal portion of the device 10 is inserted transvaginally into the patient and manipulated by the handle 20 by a user (typically by the user using just one hand on the handle 20), the sensor 26 is used to locate a uterine artery by sensing the flow of blood through the uterine artery. Because the uterine arteries are located within tissue, the sensor 26 can sense the flow of blood through the tissue. That is, the uterine artery does not necessarily need to be surgically exposed and inserted into the recessed area 24 to be sensed by the sensor 26. The sensor 26 is used to sense generally where there is blood flow and then to locate more precisely that flow by manipulating the device 10 such that the tissue near the artery is disposed within the recessed area 24 and the signal from the sensor 26 is the strongest. The ultrasound sensor 26 is disposed on the arcuate section 22, in the disclosed embodiment according to the invention. A suturing needle deployment and catch mechanism is configured to administer a suture around the located blood vessel that is disposed within the recessed area 24. Components operatively associated with the sensor 26 and the needle deployment and catch mechanism (which are discussed in further detail below) are part of the device 10 and allow a medical professional to manipulate the device 10 into and within the body of a patient to position a blood vessel within or near the recessed area 24. A suture is then placed around that located blood vessel to at least partially stop the flow of blood through the vessel, creating an ischemic condition in the target tissue (such as a fibroid or a cyst).

Instead of the sensor 26 sensing the flow of blood through tissue, the blood vessel of interest, such as the uterine artery, can be surgically exposed by a surgeon or other medical professional making a small incision in the tissue surrounding the artery such that the artery can be brought into contact with, or at least can be brought closer to, the sensor 26. This may prevent other structures in the body (e.g., one or more other vessels such as a vein) from being sensed by the sensor 26, when that or those other structures are near the artery of interest. Arterial flow will be more pronounced than venous flow. In any event, ligating a uterine vein may not lead to complications. A structure such as a ureter will be distinguishable from the uterine artery because urinary flow is non-pulsatile and much slower. Moreover, the risk of damaging the ureter is much lower compared to other structures such as a uterine vein, because the ureter is a substantial distance from the uterine artery.

Also, for the sensor 26, technologies other than ultrasound are possible. The sensor can be a sensor of another type such as an audio transmitter and/or receiver (e.g., a microphone) or a pressure transducer. As long as the sensor can be used to locate a blood vessel within the body of a patient, any type of sensor will do. An ultrasound transducer, a microphone, and a pressure transducer are just three examples of the types of sensors that can be used in accordance with the invention. Those of ordinary skill will understand how to employ any of these and a variety of other types of sensors, consistent with the illustrative description presented herein.

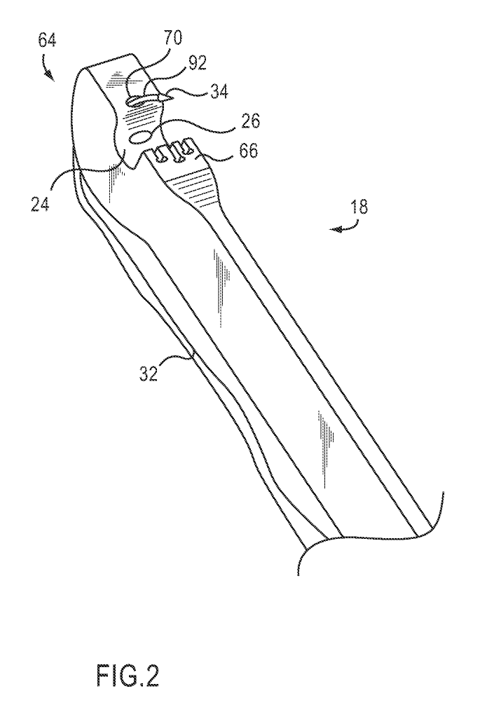

The handle 20 is disposed at a proximal end of the elongated body member 14. At the handle 20 is disposed an actuator 21 that is part of the needle deployment and catch mechanism and is for activating the needle deployment and catch mechanism. The handle 20 can take a variety of forms. For example, the handle 20 could be one of the types compatible with suturing systems available from Boston Scientific Corporation of Natick, Mass., in particular with the Capio® Push & Catch suturing system. A suture clip 30 may be coupled to the handle 20 or the elongated body member 14 and used to hold an end of one or more sutures 32 prior to placement in a patient. Generally, the needle deployment and catch mechanism includes components that extend longitudinally through the elongated body member 14 to the distal portion 18 of the device 10, where other components of the needle deployment and catch mechanism are coupled to a suturing needle 34 (shown in FIG. 1C). The needle deployment and catch mechanism moves the attached needle 34 between a retracted position and a deployed position. One possible needle deployment and catch mechanism is shown in greater detail in FIGS. 1B, 1C and 2. The needle deployment and catch comprises a needle carrier 92, a needle catch 66, and the actuator 21. The needle carrier 92 is disposed at least partially within the distal portion 18 of the elongated body member 14. In its rest or non-activated or retracted state, the needle carrier 92 remains within a passage defined by the distal portion 18, and it is movable out of that passage to move the needle 34 from one side of the recessed area 24 to the other side of the recessed area 24. The path of the needle 34 can be a semi-circle or substantially a semi-circle, and the needle carrier 92 can be curved to cause the needle 34 to travel along this curved path. Other paths are possible, such as a straight path whereby a straight or substantially straight needle travels a straight or substantially straight path across or through the recessed area 24. The needle catch 66 is disposed on the distal portion 18 and is for receiving and retaining the needle 34 when it reaches the other side of the recessed area 24. The actuator 21 is coupled to the needle carrier 92 through a series of components that extend from the proximal end of the device 10 to its working distal end.

Referring to FIG. 1B, in one embodiment, a proximal portion of the device 10 includes the handle 20, the proximal portion of the elongated body member 14, the suture clip 30, the proximal components of the ultrasound sensor 26, and the proximal components of the needle deployment and catch mechanism. For the disclosed embodiment, these proximal components of the needle deployment and catch mechanism include an actuator button 36 and a shaft 38 that together form the actuator 21. The proximal components of the needle deployment and catch mechanism also include a bearing 42 and a button end 44 that defines a hole 46 formed therein. Hole 46 is preferably formed along the central longitudinal axis of the button end 44. The bearing 42 rides along the surface of a lumen 48 that is defined by the inside diameter of the elongated body member 14. A wireform 50 is inserted into the hole 46 of the button end 44, so that the wireform 50 is coupled to the actuator button 36. A spring 52 encircles the wireform 50, abuts the button end 44, and is compressed between the button end 44 and a spring washer 54. The spring washer 54 is seated upon a center tube 56. The center tube 56 is housed by the lumen 48 and is constrained in the distal portion 18. A pusher wire 58 is attached to the wireform 50 by means of a weld, a coupling, adhesive, or other means, and is slidably disposed within a guidance sleeve 60, the sleeve 60 being disposed within the surface of a lumen 62 defined by the inside diameter of the center tube 56.

In one embodiment, the pusher wire 58 is constructed of an elastic material having “superelastic” properties. Such a material may include alloys of In—Ti, Fe—Mn, Ni—Ti, Ag—Cd, Au—Cd, Au—Cu, Cu—Al—Ni, Cu—Au—Zn, Cu—Zn, Cu—Zn—Al, Cu—Zn—Sn, Cu—Zn—Xe, Fe₃ Be, Fe₃Pt, Ni—Ti—V, Fe—Ni—Ti—Co, and Cu—Sn. In the illustrative embodiment, the superelastic material is a nickel and titanium alloy, commonly known as Nitinol® available from Memry Corp. of Brookfield, Conn. or SMA Inc. of San Jose, Calif., so chosen for its combination of properties that allow for bendability and high column strength when constrained. The ratio of nickel and titanium in Nitinol® may vary. One preferred example includes a ratio of about 50% to about 56% nickel by weight. Nitinol® also possesses shape retention properties.

The proximal components associated with the ultrasound sensor 26 include an external system 28 which is operatively associated and in communication with sensor 26. The system 28 can send energy to and receive signals from the sensor 26, by, for example, a wired connection 25 extending from the sensor 26, through the elongated body member 14, and exiting the device 10 where it is then attached to the system 28. Alternatively, the system 28 may be mounted on device 10 at the proximal end. Also, the system 28 and sensor 26 may be configured to communicate with one another via a wireless connection.

The system 28 can provide power to the sensor 26 and also control the sensor 26 by sending appropriate excitation signals to it and receiving signals from it. The system 28 also can provide a user with audio and/or visual indications and feedback corresponding to the sensor's transmissions and receptions within the body of the patient. The system 28 has processing components which allow it to process the signals from the sensor 26. The system 28 thus facilitates operation of the sensor 26 and assists the user of the device 10 in determining the position of the distal end of device 10 relative to anatomical features within the body of a patient. In one embodiment, the system 28 is an ultrasonic imaging device, such as a gray scale color two-dimensional Doppler ultrasound system, but could be a three-dimensional ultrasound system or another type of ultrasound system used in gynecological applications which allows the user to determine visually and/or aurally the location of the distal portion of the device 10, and in particular the recessed area 24, relative to a uterine artery. The user can employ the system 28 to listen for the characteristic sounds associated with blood flow through the uterine arteries and, using an increase in the magnitude of such sounds as a guide, locate the uterine artery of interest that is to be occluded.

Referring to FIGS. 1C and 2, the distal portion 18 of the elongated body member 14 includes the distal components of the ultrasound sensor 26 and needle deployment and catch mechanism. The needle catch 66 may be constructed of thin stainless steel of high temper, such as ANSI 301 full hard. The needle catch 66 may be fabricated by means of stamping, laser machining, or chemical etching, for example. In this embodiment, the operative portion 64 of the distal portion 18 has an arcuate or generally arcuate or C-like shape and defines the recessed area 24. The operative portion 64 also defines a lumen 68 therein having a needle exit port 70 at an opening adjacent recessed area 24. The needle 34 is disposed in the needle exit port 70 and is held in place by a slight friction fit, where it will stay until the needle carrier 92 is moved through the lumen 68 and thus comes into contact with the needle 34 and engages it at a receiving distal end of the needle carrier 92. Alternatively, the needle 34 can be pre-loaded onto the distal receiving end of the needle carrier 92 and friction fit onto that end of the needle carrier 92. In one embodiment, the suture 32 is attached to the needle 34, with the free end of the suture 32 extending out of a suture slot 72 and then back up to the proximal end of the device 10 where it can be held in place by the suture clip 30 as shown in FIG. 1A.

Beyond the needle carrier 92 and the needle catch 66, the distal components of the needle deployment and catch mechanism include the pusher wire 58 which is attached by welding or other means to a coupling 74. The coupling 74 is slidably disposed within a track 76. The coupling 74 is attached to a carrier wire 78, which, by virtue of its attachment to the coupling 74, is also slidably disposed within the track 76. The coupling 74 abuts a backstop washer 80 that is slidably disposed about the pusher wire 58 and is contained within a pocket 82 that includes a back wall 84, against which the backstop washer 80 rests. The track 76 terminates distally in a pocket 86 that includes a wall 88. A downstop washer 90 is slidably disposed about the carrier wire 78 and constrained within the pocket 86.

The carrier wire 78 is mechanically coupled to an extendable needle carrier 92 by welding, coupling, use of adhesives, or by other means. The needle carrier 92 is slidably disposed in the lumen 68 of the operative portion 64. A receiving port is formed at a distal end of the needle carrier 92, and this port dimensioned to releasably receive and releasably retain the non-penetrating end of the needle 34 by, for example, a friction fit. The needle carrier 92 is configured to push the needle 34 out of the needle exit port 70, through tissue disposed within the recessed area 24, and into the needle catch 66. When the user releases hand-applied depression force on the actuator 21, it springs back proximally to its biased position and thus causes the needle carrier 92 to return back into the lumen 68 through the exit port 70 from which it emerged when the actuator 21 was pushed in distally. The needle 34 gets caught and retained within the needle catch 66, but the needle carrier 92 retracts and returns back to its non-activated and retracted state within the lumen 68.

The distal components of the ultrasound sensor 26 generally include the sensor 26 itself. If hard-wired to the system 28, the distal components also include the wire(s) running back through the device 10. If wireless, the distal components include wireless transceiver equipment to allow communication with the system 28. The sensor 26 can be a transducer for emitting signals (such as ultrasonic signals, for example) and detecting return signals (such as echoes) when the distal end of the device 10 is maneuvered within the body of a patient. When the emitted sound from the ultrasound sensor 26 encounters a border between two tissues that conduct sound differently, some of the sound waves bounce back to the transducer, thus creating an echo. The echoes are analyzed (in real time, for example) by the system 28 and preferably transformed into moving pictures and/or aural information related to the anatomical features of the patient that are adjacent the sensor 26. In one embodiment, the sensor 26 thus can be configured to deliver data that can be converted by the system 28 into audio and/or video representations of locations within the body of the patient.

In general, the disclosed device 10, and other such devices in accordance with the invention, should be made of biocompatible materials and components. For example, in the disclosed embodiment of the device 10, the handle 20, the elongated body member 14, and the actuator 21 can be fabricated from extruded, molded, or machined plastic material(s) such as polypropylene, polyethylene, polycarbonate, or glass-filled polycarbonate. Other components, such as the other components of the needle deployment and catch mechanism and the needle 34, can be made of stainless steel. The material(s) used to form the suture 32 should be biocompatible too. The surgeon or other medical personnel will select the length, diameter, and characteristics of the suture to suit a particular application. Additionally, some of the mechanical components and the operation of the device 10 are similar in nature to those disclosed in U.S. Pat. Nos. 5,364,408 and 6,048,351, and U.S. Patent Application Publication No. 2004/0034372 A1 (i.e., Ser. No. 10/210,984), each of which is incorporated by reference herein in its entirety.

The device 10 is usable for both temporary and permanent occlusion of various blood vessels including one or both of the uterine arteries. That is, a device according to the invention can be used to collapse a blood vessel and hold it closed or at least partially closed for an extended period of time into the future, or a device according the invention can be used to collapse a blood vessel for some period of time but then the collapsed vessel can be released. In general, ligating a blood vessel by placing a suture around it and tightening the suture so as to stop or at least partially stop the flow of blood through that vessel and leaving the tightened suture in place around the vessel for an extended period of time may close or at least partially close off the vessel permanently even if the restricting suture is removed after that extended period of time.

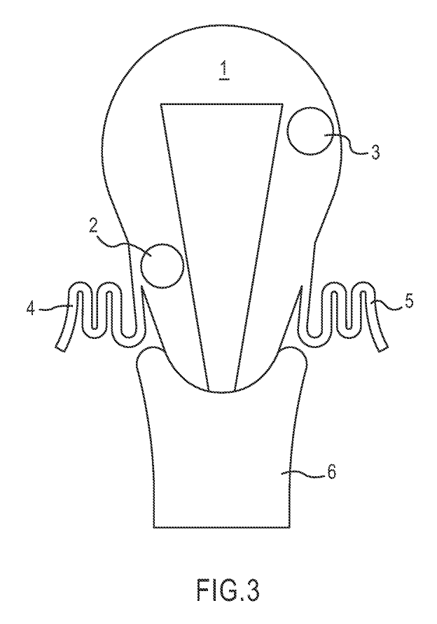

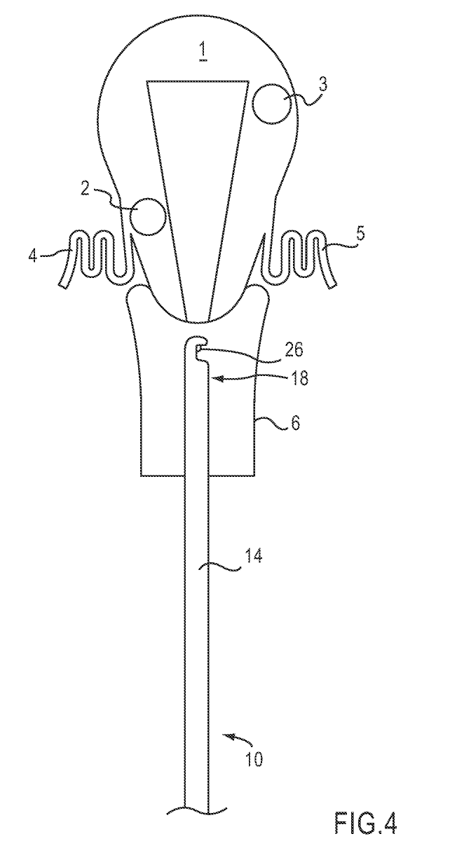

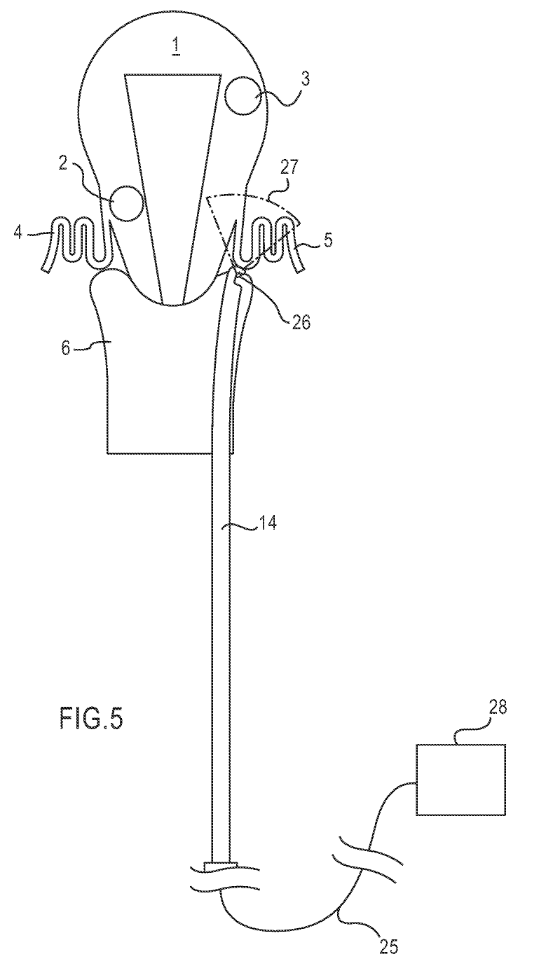

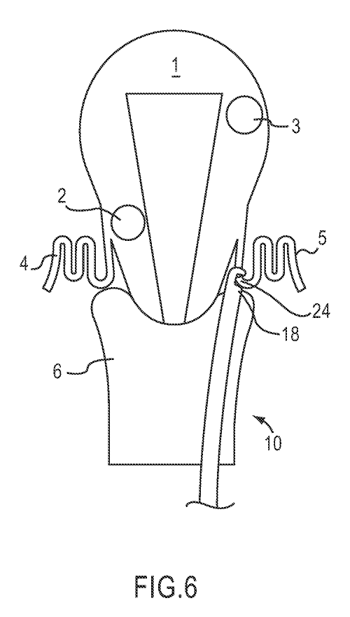

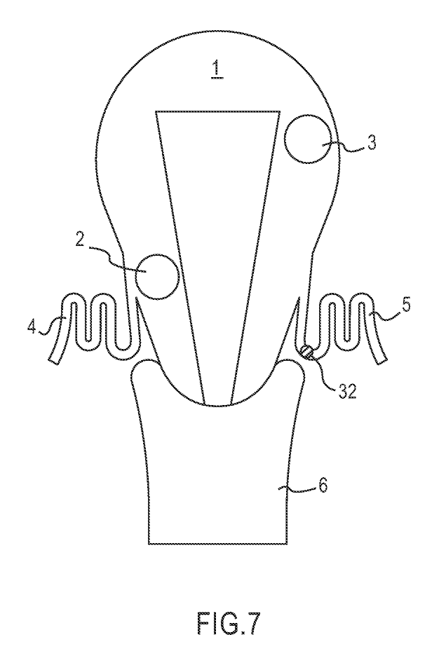

As illustrated in FIGS. 3 through 7, a patient's uterus 1 is afflicted with two representative fibroids or myomas 2 and 3. Blood is supplied to the uterus 1 primarily via the right uterine artery 4 and the left uterine artery 5. As an initial step in the procedure, the distal portion of the device 10 is introduced transvaginally and advanced through the vagina 6 toward uterus 1.

A user (such as a surgeon, physician, or other medical personnel) then locates the uterine artery of interest. For purposes of illustration, the left uterine artery 5 only is located in this exemplary use. It is located using the ultrasound sensor 26, which, in this embodiment, is configured to emit ultrasonic signals within a detection range 27. The sensor 26 communicates with the system 28 to deliver data relating to detection range 27. The system 28 processes the data received from sensor 26 to generate an image of the anatomical features within detection range 27. This process occurs in real-time so that the system 28 illustrates the anatomical features within detection range 27 as the distal portion of the device 10 (along with sensor 26 and detection range 27) are moved within the body of the patient.

In general, the device 10 is maneuvered so that the left uterine artery 5 is in the recessed area 24, and then a suture 32 is applied around the artery 5 by pushing on the button 36, which, via the coupling to the wireform 50, is coupled to the pusher wire 58 and so moves the coupling 74 along the track 76 concomitantly moving the carrier wire 78 which in turn slidably moves the needle carrier 92 through the lumen 68 towards the needle exit port 70. The user continues to push the button 36 until the needle carrier 92 advances the needle 34 (or until it receives the needle 34, if the needle 34 is not yet releasably coupled to the distal end of the needle carrier 92, and then advances the needle 34) out of the exit port 70, through tissue disposed within the recessed area 24, and into the needle catch 66. Then the user releases the button 36, and the spring 52 urges the button 36 proximally, thereby moving the pusher wire 58, the coupling 74, and the carrier wire 78 to the retracted position, which retracts the needle carrier 92 back into the lumen 68. As the needle carrier 92 moves back to the retracted position, the needle 34 releases from the end of the needle carrier 92 because the needle 34 is held and retained within the needle catch 66. The suture 32 attached to the needle 34 thus is passed through the tissue and around the artery 5.

After the suture 32 is placed, the user withdraws the device 10 transvaginally from the patient. The user then detaches the suture 32 from the needle 34 (which is retained within the needle catch 66) and the user ties a knot in the suture 32 and uses a knot pusher 96 at the distal end of the device 10 to push the knot into the patient and around the artery 5 to ligate the artery 5 and occlude it by at least partially stopping blood from flowing through the artery 5 and preferably stopping the blood flow through the artery 5 entirely.

A surgeon, physician, or other medical professional can remove the suture 32 after a period of time has elapsed during which benefits have been realized by the patient, such as after successful treatment of uterine fibroids. Alternatively, the suture 32 may be formed of a material that is biodegradable, thus avoiding any further procedures relating to removal thereof. Unlike a transvaginal clamp, the suture 32 can be installed by device 10 to treat uterine fibroids without impeding the patient's mobility.

The embodiments of the invention shown and described herein permit a surgeon, physician, or other medical professional to locate and occlude blood vessels within the body of a human patient or other mammal. Having described such embodiments of the invention, various alterations, modifications, and improvements will be apparent to those of ordinary skill. Such alterations, modifications, and improvements are intended to be within the spirit and scope of the invention. The invention is not limited just to the foregoing disclosure of certain embodiments of the invention.

What is claimed is:

1. A medical device, comprising:
a) an elongated member including a distal portion configured and dimensioned for accessing anatomical regions, the distal portion including a recessed area;
b) a sensor disposed on the distal portion within the recessed area for sensing a blood vessel disposed within the recessed area; and
c) an occluder operatively associated with the distal portion and configured for at least partially stopping the flow of blood through the sensed blood vessel disposed within the recessed area.

2. The device of claim 1 wherein the occluder comprises a suture placement mechanism.

3. The device of claim 2 wherein the suture placement mechanism comprises a needle deployment and catch mechanism.

4. The device of claim 3 wherein the needle deployment and catch mechanism comprises:
i) a needle carrier disposed at least partially within the distal portion of the elongated body member, the needle carrier movable out of the distal portion to move a needle attachable to the needle carrier from one side of the recessed area to the other side of the recessed area;
ii) a needle catch disposed on the distal portion for receiving and retaining the needle; and
iii) an actuator coupled to the needle carrier and disposed at a proximal end of the elongated member for moving the needle carrier.

5. The device of claim 4 wherein the actuator comprises a button actuatable by a hand of a user and a shaft extending from the button.

6. The device of claim 4 further comprising a suture attached to the needle.

7. The device of claim 1 wherein the sensor comprises an ultrasound sensor.

8. The device of claim 1 wherein the sensor comprises a microphone.

9. The device of claim 1 wherein the sensor comprises a pressure transducer.

10. A method of locating and occluding a blood vessel, comprising:
a) providing a medical device comprising
i) an elongated member including a distal portion configured and dimensioned for accessing anatomical regions, the distal portion including a recessed area;
ii) a sensor disposed on the distal portion within the recessed area for sensing a blood vessel disposed within the recessed area; and
iii) an occluder operatively associated with the distal portion and configured for at least partially stopping the flow of blood through the blood vessel disposed within the recessed area;
b) maneuvering the medical device so that the blood vessel to be occluded is within the recessed area; and
c) stopping at least partially the flow of blood through the blood vessel using the medical device.

11. The method of claim 10 wherein the occluder comprises a suture placement mechanism and the stopping step includes placing a suture around the blood vessel to be occluded.

12. The method of claim 10 wherein the sensor comprises an ultrasound sensor and the maneuvering step includes using the ultrasound sensor to sense the flow of blood through the blood vessel.

13. A medical device, comprising:
a) means for accessing anatomical regions of a patient and for receiving at least a blood vessel of the patient;
b) means for sensing the blood vessel; and
c) means for at least partially stopping the flow of blood through the sensed blood vessel.

14. The device of claim 13 wherein the occluding means comprises a suture placement mechanism.

15. The device of claim 14 wherein the suture placement mechanism comprises a needle deployment and catch mechanism.

16. The device of claim 15 wherein the needle deployment and catch mechanism comprises a needle carrier and a needle catch.

17. The device of claim 16 wherein a needle is removably couplable to the needle carrier and a suture is attached to the needle.

18. The device of claim 13 wherein the sensing means comprises an ultrasound transducer.

19. The device of claim 13 wherein the sensing means comprises a microphone.

20. The device of claim 13 wherein the sensing means comprises a pressure transducer.
